(19) United States
(12) Patent Application Publication
(10) Pub. No.: US 2026/0076639 A1
(43) Pub. Date: Mar. 19, 2026

(54) X-RAY IMAGING SYSTEMS AND DETECTORS

(71) Applicant: VAREX IMAGING CORPORATION, Salt Lake City, UT (US)

(72) Inventors: Marcelo C. Costa, Draper, UT (US); Carlo Tognina, Salt Lake City, UT (US)

(73) Assignee: VAREX IMAGING CORPORATION, Salt Lake City, UT (US)

(21) Appl. No.: 19/109,843

(22) PCT Filed: Nov. 16, 2023
(86) PCT No.: PCT/US2023/080170

§ 371 Date: Mar. 7, 2025

Related U.S. Application Data
(60) Provisional application No. 63/384,406, filed on Nov. 18, 2022.

(51) Int. Cl. A61B 6/00; A61B 6/10; A61B 6/42

(52) U.S. Cl. CPC A61B 6/542; A61B 6/107; A61B 6/4233; A61B 6/4291; A61B 6/4452; A61B 6/547

(57) ABSTRACT

Embodiments include an x-ray detector, comprising: a housing; an imaging array disposed within the housing; circuitry configured to generate an image in response to the imaging array; and an automatic exposure control (AEC) chamber disposed within the housing, separate from the imaging array, and on a side of the imaging array opposite to the circuitry.

### BACKGROUND

X-ray systems may include automatic exposure control (AEC) components. These AEC components may be mounted in a particular, fixed location. Thus, an operation using the AEC components may be limited to that fixed location.

### BRIEF DESCRIPTION OF THE DRAWINGS

FIG. 1 is a block diagram of a mobile detector including AEC according to some embodiments.

FIG. 2A is a block diagram of a mobile detector including AEC embedded in a front panel according to some embodiments.

FIG. 2B is a block diagram of a mobile detector including AEC behind a front panel according to some embodiments.

FIG. 2C is a block diagram of a mobile detector including AEC behind a front panel according to some embodiments.

FIG. 2D is a block diagram of a mobile detector including AEC behind a front panel according to some embodiments.

FIG. 2E is a block diagram of a mobile detector including AEC behind a front panel with resilient material according to some embodiments.

FIG. 3A is a block diagram of a mobile detector including AEC chamber and an AEC preamplifier according to some embodiments.

FIG. 3B is a block diagram of a mobile detector including AEC chamber coupled to detector circuitry according to some embodiments.

FIG. 4 is a block diagram of a mobile detector including digital AEC according to some embodiments.

FIG. 5 is a block diagram of a mobile detector including AEC and a grid according to some embodiments.

FIG. 6 is a block diagram of an x-ray imaging system according to some embodiments.

FIG. 7 is a block diagram of an x-ray imaging system with multiple detector locations according to some embodiments.

FIG. 8 is a block diagram of an x-ray imaging system with multiple detector locations according to some embodiments.

### DETAILED DESCRIPTION

Conventional fixed installations for radiography applications have a bucky, an automatic exposure control (AEC) chamber and pre-amplifier, and a grid for optimal image quality. When multiple locations are present in the fixed installation, each of those locations will need its own bucky, AEC chamber and pre-amplifier, and grid. For example, a radiology room may include a table and a stand as locations for x-ray imaging. An x-ray generator attached to a crane may be movable to direct x-rays to either location. However, each of the stand and the table will need its own bucky, AEC chamber and pre-amplifier, and grid even if a single detector is moved from location to location.

The fixed installations of AEC chambers prevent the usage of AEC in mobile applications. For example, a patient may be unable to be moved to the radiology room. A mobile detector and x-ray generator may be moved to the patient; however, the system will not have AEC. As a result, AEC and its benefits will not be available when imaging the patient.

As will be described in further detail below, in some embodiments, a mobile detector may eliminate a need for the bucky, AEC chamber, and grid from the system. The AEC chamber and grid functionality may be performed by the mobile detector. In addition to eliminating duplicate parts, embodiments may allow for AEC to be available in mobile applications.

A variety of detectors 100a, 100b, etc. are described below. These detectors may be referred to collectively as detectors 100. A variety of systems 200a, 200b, etc. are described below. These systems may be referred to collectively as systems 200.

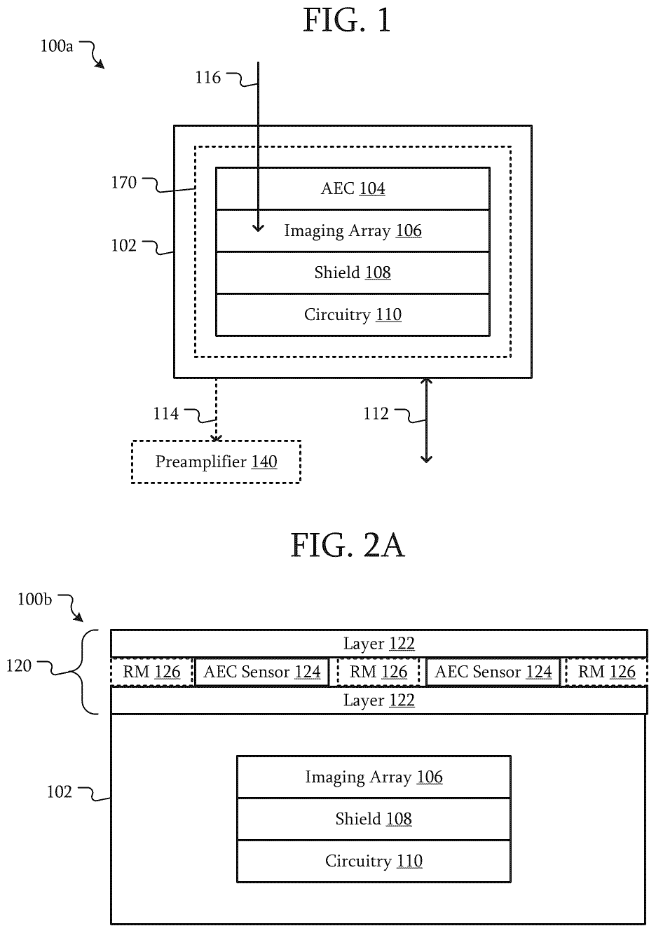

FIG. 1 is a block diagram of a mobile detector including AEC according to some embodiments. The detector 100a includes a housing 102, an AEC chamber 104, an imaging array 106, a shield 108, and circuitry 110. The detector 100a and the components are disposed such that imaging array 106 is configured to receive incoming radiation 116 (e.g., incident x-rays) through the AEC chamber.

The housing 102 is a structure enclosing the AEC chamber 104, the imaging array 106, the shield 108, and the circuitry 110. Other components may be enclosed by the housing 102. In some embodiments, the housing 102 fully encloses the various components.

The AEC chamber 104 includes AEC sensors and circuitry configured to transform incoming radiation into an electrical signal or AEC signal. The AEC signals may be used to determine a dose and/or whether to terminate an exposure. The AEC sensors may include solid state sensors, ionization chambers, or the like.

The imaging array 106 includes an array of pixels configured to transform incoming radiation 116 into a two-dimensional image. The imaging array 106 may include direct-conversion sensors, photon counters, indirect-conversion sensors, scintillators, or the like.

The shield 108 is a structure configured to reduce radiation reaching the circuitry 110. For example, the shield 108 may include lead, tin, or other materials that may have a relatively higher absorption of x-rays.

As will be described in further detail below, the AEC chamber 104 including the AEC sensors may be disposed in various locations separate from the imaging array 106, such as in or on a front plate or front panel (not illustrated) of the housing 102, behind the front plate, on the imaging array 106, or the like.

Integration of the AEC sensors with the components of the detector 100a may reduce the costs of the use of an AEC chamber 104. As the AEC chamber 104 is disposed in the detector 100a, when the detector 100a is moved from location to location, such as from a stand in a radiology room to a table, a separate AEC chamber is not needed in those locations. In addition to the reduced number of components, costs and complexity for an original equipment manufacturer (OEM) using the detector 100a may be reduced. An OEM may no longer need to consider buying a separate AEC chamber. An OEM may have a shorter integration time as the detector 100a may have a unified software interface to both the imaging array 106 and the AEC chamber 104. In addition, the detector 100a may be configured to resolve scatter and eliminate a requirement for an external grid.

Moreover, the presence of the AEC chamber 104 in the detector 100a allows AEC to be used in mobile applications outside of the radiology room. AEC in mobile applications may allow for improved workflow, image quality, and lower dose for patients and users, or the like.

In some embodiments, the output of the AEC chamber 104 may be provided to a preamplifier 140. In some embodiments, the preamplifier 140 is disposed outside of the detector 100a. However, in other embodiments, the preamplifier 140 or the function of the preamplifier 140 may be within the detector 100a.

In some embodiments, the housing 102, portions of the housing 102, and other conductive structures, foils, or the like within the housing 102 may form a faraday cage 170 around at least the AEC chamber 104 and up to potentially all internal components of the detector 100a. The AEC chamber 104 may be disposed within the faraday cage 170. The AEC chamber 104 may generate signals that are on the order of millivolts (mV) or microvolts (μV). According, the signals may be particularly susceptible to noise. By placing the AEC chamber 104 within the faraday cage 170, the effect of noise may be reduced. In particular, as will be described in further detail below the preamplifier 140 may be disposed within the faraday cage 170. Thus, the signals from the AEC chamber 104 may be amplified before becoming more susceptible to noise outside of the faraday cage 170.

In some embodiments, the signals from the AEC chamber 104 may be used to begin an exposure. Thus, the AEC chamber 104 may be used to perform an automatic exposure detection (AED) operation. As a result, the detector 100a may not be synchronized with an x-ray generator (not illustrated), but still able to determine the onset of x-rays from the x-ray generator to begin an exposure. This may reduce a cost of a system including the detector 100a, simplify an installation, or the like.

The use of the AEC chamber 104 to begin an exposure may be more robust than using the imaging array 106 to detect x-rays. Sensors within the imaging array 106 may be more sensitive to impacts, mechanical disturbances, or the like. The impacts may result in a false detection of x-rays. An AEC chamber 104 may be less susceptible to such false detections, leading to a more reliable system.

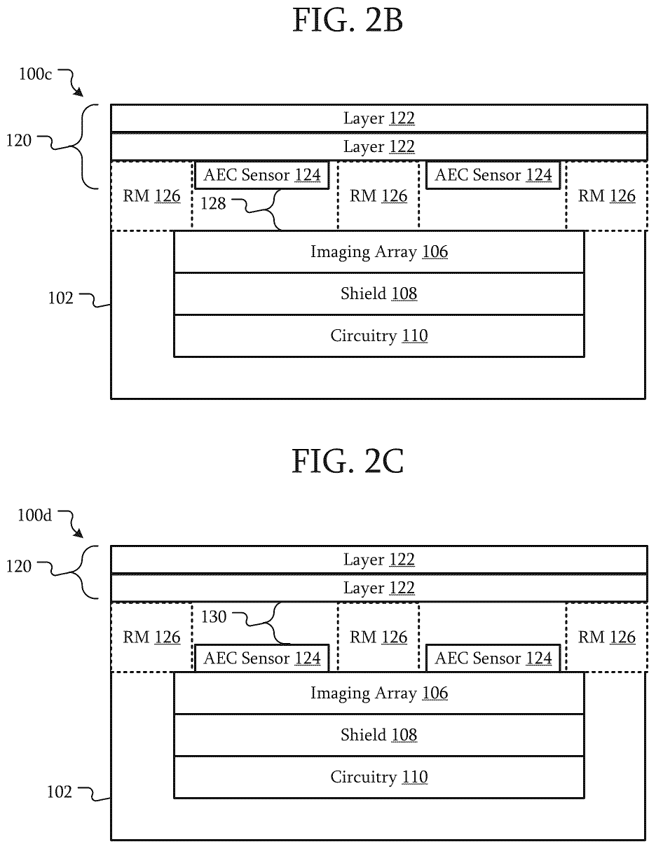

FIG. 2A is a block diagram of a mobile detector including AEC embedded in a front panel according to some embodiments. In some embodiments, the detector 100b may be similar to the detectors 100 described herein. However, the housing 102 includes a front panel 120 (also referred to as a front plate). The front panel 120 may include laminated layers 122 such as carbon fiber layers, fiberglass layers, or the like. AEC sensors 124 of the AEC chamber 104 are disposed within the layers 122 of the front panel 120. In some embodiments, the AEC sensor 124 can be positioned between a top or first layer 122 and a bottom or second layer 122.

In some embodiments, the layers 122 conform to the shape of the AEC sensors 124. However, in other embodiments, a resilient material 126 such as foam may be disposed in substantially the same plane as the AEC sensors 124 and with substantially the same or greater thickness. The resilient material 126 may not be present over the AEC sensors 124. As a result, the AEC sensors 124 may be at least in part isolated from mechanical stresses applied to the front panel 120. For example, a weight of a patient may cause the front panel 120 to flex. The flexing may have a reduced effect on the AEC sensors 124 due to the resilient material 126.

In implementations with a separate AEC chamber, a rigid structure may be present such as a table or a bucky that is between a patient and the AEC chamber. However, with the detector 100b or the like, such rigid structures may not be present. As a result, the detector 100b may include various features as described herein to mitigate flexing that may occur.

FIG. 2B is a block diagram of a mobile detector including AEC behind a front panel according to some embodiments. In some embodiments, the detector 100c may be similar to the detectors 100 described herein. However, the AEC sensors 124 may be disposed behind the front panel 120. In particular, the AEC sensors 124 are disposed on the front panel 120. The AEC sensors 124 may be separated from the imaging array 106 by a gap 128. In some embodiments, the gap 128 may be about 0.2 to about 1 mm or more. In some embodiments, a resilient material 126 may be disposed between the front panel 120 and the imaging array 106. The resilient material 126 may be disposed around the AEC sensors 124 similar to that described above.

FIG. 2C is a block diagram of a mobile detector including AEC behind a front panel according to some embodiments. In some embodiments, the detector 100d may be similar to the detectors 100 described herein. However, the AEC sensors 124 may be disposed on the imaging array 106. The AEC sensors 124 may be separated from the front panel by a gap 130. In some embodiments, the gap 130 may be about 0.2 to about 1 mm or more. In some embodiments, a resilient material 126 may be disposed between the front panel 120 and the imaging array 106. The resilient material 126 may be disposed around the AEC sensors 124 similar to that described above.

FIG. 2D is a block diagram of a mobile detector including AEC behind a front panel according to some embodiments. In some embodiments, the detector 100e may be similar to the detectors 100 described herein. However, the AEC sensors 124 are disposed within the imaging array 106.

FIG. 2E is a block diagram of a mobile detector including AEC behind a front panel with resilient material according to some embodiments. In some embodiments, the detector 100f may be similar to the detectors 100 described herein. However, the AEC sensors 124 may be disposed between a resilient material 126 and the imaging array 106. The resilient material 126 may be configured to apply an amount of compressive force to the AEC sensors 124 due to compression by the front panel 120. The resilient material 126 may be selected to have composition, thickness, resilience, or the like to apply a predetermined force to the AEC sensors 124. The predetermined force may keep the AEC sensors 124 in place over an expected operation or lifetime of the detector 100f. The predetermined force may be sufficient to keep the AEC sensors 124 in place while being less than an amount that would damage the AEC sensors 124. In addition, the resilient material 126 may be selected to have a lower x-ray attenuation. Examples of the resilient material 126 include foam, rubber, or the like.

In some embodiments, the resilient material 126 may extend across the entire imaging array 106 or beyond the edges of the imaging array 106. In some embodiments, the resilient material 126 may be within one to all of the edges of the imaging array 106 but still overlap with the AEC sensors 124.

While the use of the resilient material 126 is an example of how to maintain the AEC sensors 124 in a particular position, in other embodiments, other techniques may be used. For example, permanent adhesives, releasable adhesives, brackets, clamps, or the like may be used to attach the AEC sensors 124 to the imaging array 106, maintain a relative position between the AEC sensors 124 and the imaging array 106, or the like.

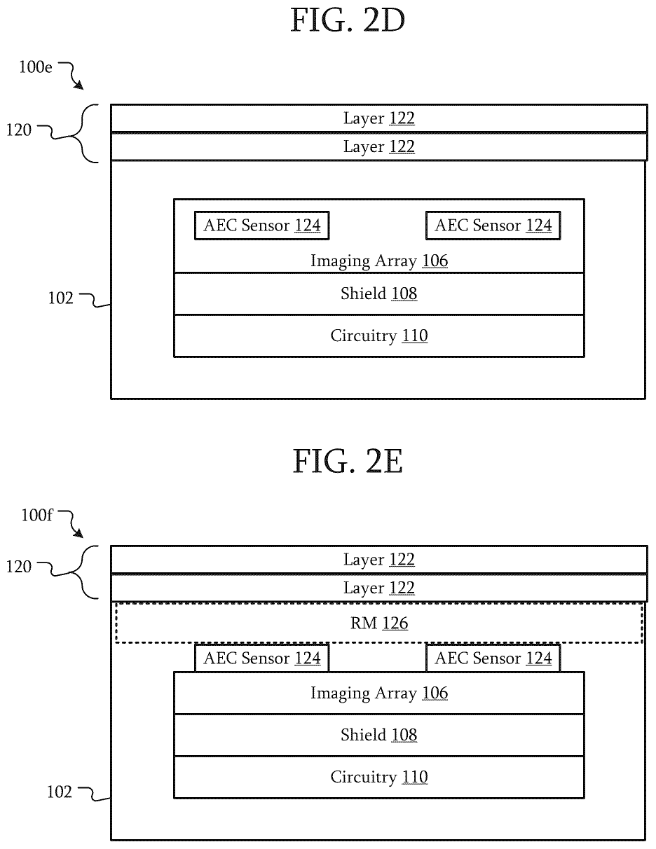

FIG. 3A is a block diagram of a mobile detector including AEC chamber and an AEC preamplifier according to some embodiments. In some embodiments, the detector 100g may be similar to the detectors 100 described herein. The detector 100g includes an AEC preamplifier 140. The preamplifier 140 is disposed within the housing 102. The preamplifier 140 may be disposed within the faraday cage 170. The preamplifier 140 is coupled to the AEC chamber 104 and is configured to receive signals from the AEC chamber 104. In some embodiments, the output of the preamplifier 140 may be a signal 114 that is transmitted to an x-ray generator to control whether the x-ray exposure should stop. Thus, the detector 100g may be a drop-in replacement where a separate AEC chamber may have been used. In some embodiments, the output of the preamplifier 140 may be input to the circuitry 110. AEC signals may be output as signals 112 to a control system, x-ray generator, or the like.

In some embodiments, the preamplifier 140 may be closer to the AEC chamber 104 than would otherwise be achieved with a separate AEC chamber. As a result, the effect of the environment on the signals from the AEC chamber 104 may be reduced, improving signal quality.

FIG. 3B is a block diagram of a mobile detector including AEC chamber coupled to detector circuitry according to some embodiments. In some embodiments, the detector 100h may be similar to the detectors 100 described herein. The AEC chamber 104 is electrically coupled to the circuitry 110. In some embodiments, the AEC chamber 104 may be electrically coupled to the circuitry 110 through pogo pins, cables, wires, transmission lines, or the like.

In some embodiments, the preamplifier 140 is part of the circuitry 110. The circuitry 110 may be configured to perform all of the signal conditioning associated with signals from the AEC chamber 104. The signal 114 or portion of the signals 112 may be in a format suitable for use by an x-ray generator such that the x-ray generator may disable the production of x-rays. In other embodiments, less than all of such signal processing may be performed by the circuitry 110. In some embodiments, the circuitry 110 is configured to generate a digital pulse, a ramp signal, or the like to start and/or stop the exposure using the x-ray generator. In some embodiments, the circuitry 110 may be configured to provide power to the AEC chamber 104, debounce signals from the AEC chamber 104, or the like.

In some embodiments, the circuitry 110 may include comparators, amplifiers, or the like to perform particular operations based on the signals from the AEC chamber 104. For example, one or more signals from the AEC chamber 104 may be compared with a threshold to determine whether to start an exposure. An image may be generated from the imaging array 106 based on the comparison.

In some embodiments, the communication of the AEC signals from the detector 100h may be through a variety of communication techniques. For example, the circuitry 110 may be configured to transmit AEC signals through a low latency digital wireless link, a wired tether with digital and/or analog signals, or the like. The signals 112 are representative of signals transmitted through such media.

In some embodiments, the circuitry 110 is configured to receive configuration inputs to define the AEC configuration. For example, the circuitry 110 may be configured to perform the calibration of the AEC chamber 104.

As described above, the preamplifier 140 may be disposed within the detector 100 and disposed within a faraday cage 170 of the detector. Thus, the signals input to the preamplifier 140 from the AEC chamber 104 may be less susceptible to noise.

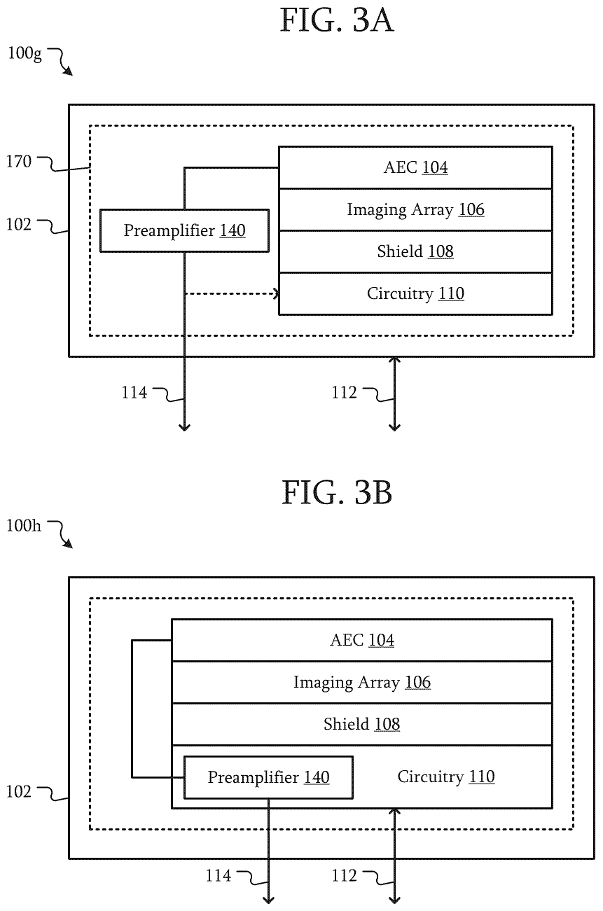

FIG. 4 is a block diagram of a mobile detector including a digital AEC 150 according to some embodiments. In some embodiments, the detector 100i may not include a separate physical AEC chamber. The circuitry 110 may be configured to use the imaging array 106 to perform a digital AEC function.

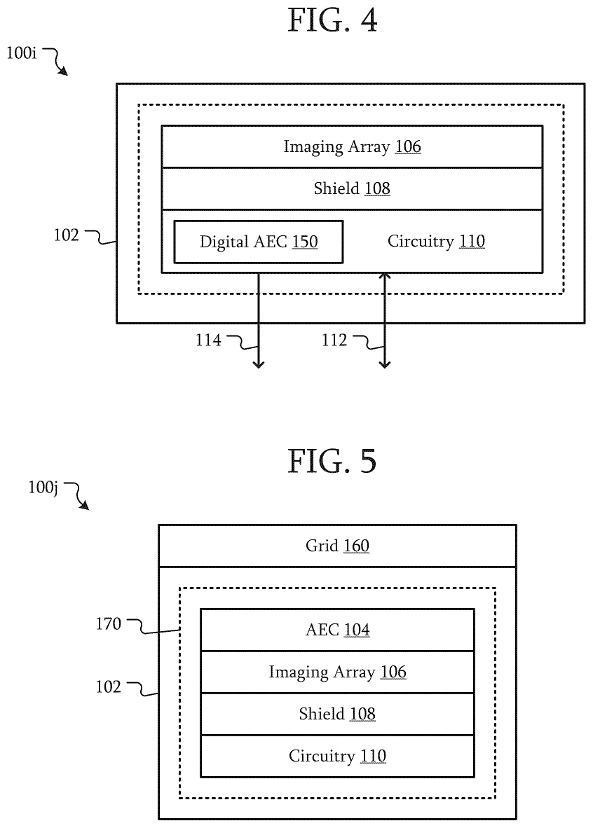

FIG. 5 is a block diagram of a mobile detector including AEC and a grid according to some embodiments. The detector 100j may be similar to the various detectors 100a-100i with AEC as described above, or the like. A grid 160 is disposed on the detector 100j. In some embodiments, the grid 160 includes a series of lead lines extending from one side of the detector 100j to the other. The lead lines may help with backscatter from the detector 100j.

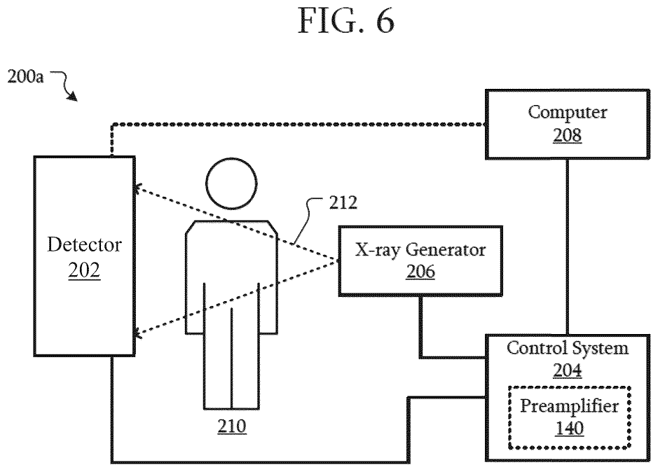

FIG. 6 is a block diagram of an x-ray imaging system according to some embodiments. The system 200a includes a detector 202, a control system 204, an x-ray generator 206, and a computer 208. The detector 202 may include any of the detectors 100 described above or the like. However, in other embodiments, the detector 202 may include different types of detectors.

In some embodiments, the x-ray generator 206 is configurable to project x-rays 212 towards the detector 202. An object 210 such as a patient may be disposed in the path of the x rays 212 to generate an image. The x-ray generator 206 may include an x-ray tube, power supplies, high voltage generators, or the like.

In some embodiments, the control system 204 may present a unified interface to the computer 208. The system 200a may be installed with a variety of different types of x-ray generators 206, detectors 202, or the like. In some embodiments, if the detector 202 is a detector 100 as described above, the AEC chamber is integrated with the detector 202. The control system 204 may be configured to communicate with and control the detector 202 and the x-ray generator 206. An installer installing the system 200a may only need to configure the computer 208 to communicate with the control system 204. The control system 204 handles the communication and control between the other components of the system 200a. Thus, the installer no longer needs to be concerned with the configuration and interaction between different types of x-ray generators 206 and detectors 202. The control system 204 may present a single interface regardless of the particular type or manufacturer of the x-ray generator 206.

In some embodiments, the AEC signals that would otherwise have been connected to the x-ray generator 206 from a separate AEC chamber (not illustrated) may be instead provided by the control system 204. The control system 204 may interact with the detector 202 to receive the AEC signals in whatever form the detector 202 may provide as described above. The control system 204 may then pass those AEC signals or transform the signals into an appropriate format for the x-ray generator 206 such that the operation of the detector 202 and the x-ray generator 206 may control the dose as desired. For example, the system 200a may have a response time that is faster than about 10 milliseconds (ms) from the x-ray generator of AEC signals to a resulting change in state of the x-ray generator 206. In some embodiments, the control system 204 may be configured to control other aspects of an AEC chamber of the detector 202. For example, the control system 204 may be configured to control calibration of the AEC chamber.

In some embodiments the control system 204 may be configured to synchronize the detector 202 and x-ray generator 206 operation. For example, the control system 204 may be configured to synchronize the generation of x-rays from the x-ray generator 206 and the capture of an image or video by the detector 202 in response to the x-rays. The control system 204 may be configured to synchronize the AEC signals with the x-ray generator 206.

In some embodiments, the detector 202 may be configured to perform grid suppression if a physical grid is present on the detector 202, such as in detector 100j, or in the system 200a. In some embodiments, the detector 202 may be configured to perform lag correction.

In some embodiments, the control system 204 may include a preamplifier 140 configured to amplify AEC signals from an AEC chamber 104 of the detector 202. The preamplifier 140 may operate similarly as described above when integrated with or separate from the detector 202. In some embodiments, a preamplifier 140 may be part of the detector 202 in addition to the preamplifier 140 of the control system 204 and may be the same or different from the preamplifier 140 of the control system 204.

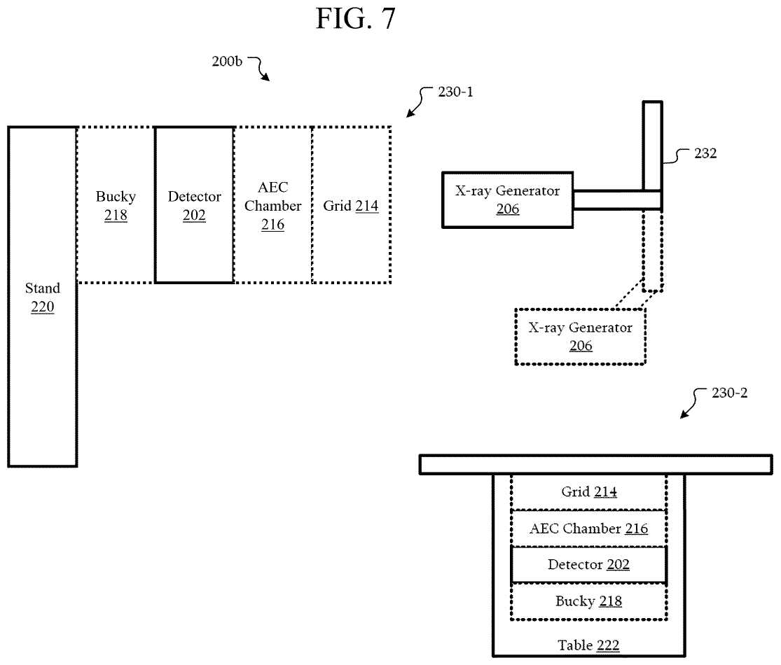

FIG. 7 is a block diagram of an x-ray imaging system with multiple detector locations according to some embodiments. The system 200b may be similar to other systems 200 described herein. A portion of the x-ray system 200b is illustrated and the x-ray system 200b may include other components similar to other x-ray systems 200. In some embodiments, the system 200b may include multiple locations 230. Here two locations 230-1 and 230-2 are illustrated as examples.

In each location 230, a grid 214, an AEC chamber 216, and a bucky 218, illustrated with dashed lines, are not present. The grid 214 and AEC chamber 216, and bucky 218 are illustrated to show the components that are no longer needed for operation. The detector 202 need only be mounted on the corresponding structure, such as a tray, of the location 230. As described above, the detector 202 may include the functionality provided by the grid 214 and the AEC chamber 216 that are not present. For example, location 230-1 may include a stand 220. The detector 202 may be mounted on the stand 220 without a bucky 218. Similarly, the location 230-2 may include a table 222. The detector 202 may be mounted on the table 222 without a bucky 218. Each location 230 may include a simple tray or other structure to hold the detector 202 in place, prevent damage such as scratches on the detector 202 surface, or the like. Moving parts of the bucky 218, shielding for the electronics of the bucky 218, UL certification of the components, or the like may be eliminated. Alignment to the grid 214 may be eliminated. These eliminations of components or processes may reduce costs associated with the system 200b.

The x-ray generator 206 may be mounted on a crane 232 that is configured to move the x-ray generator 206 to be in the orientation appropriate for the particular location 230 where the detector 202 is mounted. The x-ray generator 206 is illustrated in one position with solid lines and an alternate position with dashed lines. Depending on the particular desired application, the detector 202 may be moved between the locations 230 and moved to other locations.

In some embodiments, the detector 202 may be configured to perform scatter correction. As a grid 214 may not be present, including not being present on the detector 202, the detector 202 may be configured to perform scatter correction on the resulting image to reduce or eliminate an effect of the absence of the grid 214. In other embodiments, the control system 204 may be configured to perform the scatter correction on an image or images from the detector 202.

When using a system 200b, the cost and complexity of integrating an AEC chamber, preamplifier, bucky, grid, or the like may be reduced or eliminated.

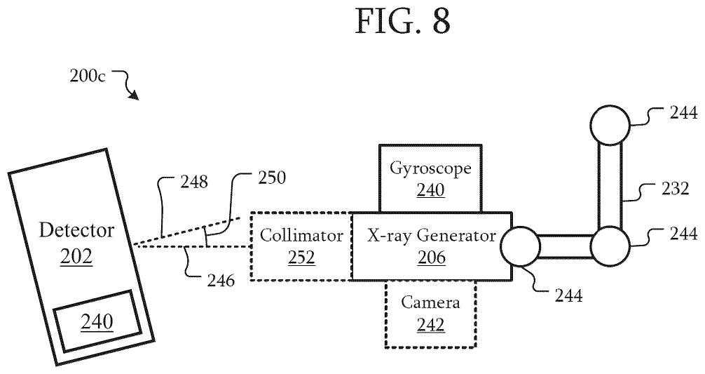

FIG. 8 is a block diagram of an x-ray imaging system with multiple detector locations according to some embodiments. The system 200c may be similar to other systems 200 described herein. A portion of the x-ray system 200c is illustrated and the x-ray system 200b may include other components similar to other x-ray systems 200. In some embodiments, the detector 202 and the x-ray generator 206 may each include a gyroscope 240. The gyroscope 240 may be configured to determine a relative rotation in one or more axes of the respective device. The gyroscope 240 associated with the x-ray generator 206 may be mounted on x-ray generator 206, on the collimator 252, or the like. In some embodiments, the crane 232 may include sensors 244 configured to sense the relative motion of components of the crane 232. The sensors 244 may be transformed into an orientation of the x-ray generator 206 in place of or in addition to the gyroscope 240.

Using the orientation information of the detector 202 and the x-ray generator 206, the detector 202 and the x-ray generator 206 may be oriented so that an angle 250 between a major axis 246 of the x-rays generated by the x-ray generator 206 and the axis 248 of the detector 202 that is perpendicular to the imaging array 106 may be set as desired or minimized. In some embodiments, the control system 204 may be configured to automatically activate actuators of the crane 232 to orient the x-ray generator 206 so that the axes 246 and 248 are substantially parallel. In other embodiments, the angle 250 or other alignment information may be presented by the control system 204 to an operator of the computer 208. As a result, an image quality may be improved. As the angle 250 between the major axis 246 and the axis 248 increases, an image quality of an image generated by the imaging array 106 may be reduced. Minimizing the angle 250 may increase the image quality. In some embodiments, the angle 250 or information based on the angle may be reported to an operator.

In some embodiments, a camera 242 may be coupled to the x-ray generator 206. The control system 204 may be configured to use an image or video from the camera 242 to determine if a patient moves and inform an operator of the computer 208. In other embodiments, the control system 204 may be configured to use an image or video from the camera 242 to select a portion of the object 210, such as a particular portion of the anatomy of a patient. In other embodiments, the control system 204 may be configured to use an image or video from the camera 242 to determine the source-to-image distance (SID) of the x-ray generator 206 and detector 202. The control system 204 may be configured to use actuators of the crane 232 to achieve a desired SID and/or present such information to an operator of the computer 208 and/or allow the operator to set a desired SID. In some embodiments, the control system 204 may be configured to use adjust a collimator 252 based on an image or video from the camera 242. For example, the control system 204 may be configured to adjust the collimator 252 so that the x-rays emitted from the x-ray generator 206 correspond to the imaging area of the imaging array 106 of the detector 202.

Although various detectors 100, systems 200, or the like have been described above with respect to radiography, other embodiments may be used in fluoroscopic applications.

Some embodiments include an x-ray detector 100, 100a-j, comprising: a housing 102; an imaging array 106 disposed within the housing 102; circuitry 110 configured to generate an image in response to the imaging array 106; and an automatic exposure control (AEC) chamber 104 disposed within the housing 102, separate from the imaging array 106, and on a side of the imaging array 106 opposite to the circuitry 110.

In some embodiments, the housing 102 comprises a front panel 120.

In some embodiments, the AEC chamber 104 is disposed within the front panel 120.

In some embodiments, the x-ray detector further comprises a layer of resilient material 126 disposed within the front panel 120; wherein sensors 124 of the AEC chamber 104 are disposed within a plane of the layer of the resilient material 126 within the front panel 120.

In some embodiments, the AEC chamber 104 is separated from the imaging array 106 by a gap.

In some embodiments, the AEC chamber 104 is disposed on the imaging array 106.

In some embodiments, the x-ray detector further comprises a layer of resilient material 126 disposed between the AEC chamber 104 and the front panel 120.

In some embodiments, the x-ray detector further comprises a shield disposed between the imaging array 106 and the circuitry 110.

In some embodiments, the x-ray detector further comprises an AEC preamplifier 140 coupled to the AEC chamber 104.

In some embodiments, the AEC preamplifier 140 is separate from the circuitry 110.

In some embodiments, the AEC preamplifier 140 is integrated with the circuitry 110.

In some embodiments, the x-ray detector further comprises a faraday cage surrounding at least the AEC chamber 104 and the AEC preamplifier 140.

In some embodiments, the x-ray detector further comprises a grid disposed on an exterior surface of the housing 102.

In some embodiments, the circuitry 110 is configured to perform scatter correction on the image.

In some embodiments, the circuitry 110 is further configured to operate in response to an AEC signal from the AEC chamber 104.

In some embodiments, the circuitry 110 is further configured begin an exposure to generate the image in response to the AEC signal from the AEC chamber 104.

Some embodiments include a method comprising: generating an automatic exposure control (AEC) signal from an AEC chamber 104 within a housing 102 and separate from an imaging array 106 within the housing 102 and generating an image using the imaging array 106 within the housing 102 in response to the AEC signal.

In some embodiments, generating the image comprises beginning an exposure associated with the image in response to the AEC signal.

In some embodiments, generating the image comprises ending an exposure associated with the image in response to the AEC signal.

Some embodiments include an x-ray system, comprising: an x-ray detector 100, 100a-j including an automatic exposure control (AEC) chamber disposed within the x-ray detector 100, 100a-j and separate from an imaging array 106 of the x-ray detector 100, 100a-j; an x-ray generator; and a control system; wherein the control system is configured to: receive AEC signals from the x-ray detector 100, 100a-j; and change an operation of the x-ray generator in response to the AEC signals.

In some embodiments, the control system comprises an AEC preamplifier configured to receive AEC signals from the x-ray detector 100, 100a-j; and the control system is configured to change the operation of the x-ray generator in response to the AEC signals received by the AEC preamplifier.

In some embodiments, the x-ray detector 100, 100a-j comprises any of the x-ray detectors 100, 100a-j of claims 1-16.

In some embodiments, the x-ray generator is movable to project x-rays towards either of a first location and a second location; the x-ray detector 100, 100a-j is movable between the first location and the second location; and the x-ray detector 100, 100a-j is configured to provide AEC signals in response to the AEC chamber 104 in both locations.

In some embodiments, the first location does not include a grid; and the second location does not include a grid.

In some embodiments, the x-ray detector 100, 100a-j further comprises a gyroscope 240 disposed within the x-ray detector 100, 100a-j; and the control system is configured to provide information related to an orientation of the x-ray detector 100, 100a-j relative to the x-ray generator based on the gyroscope 240 of the x-ray detector 100, 100a-j.

In some embodiments, the gyroscope 240 of the x-ray detector 100, 100a-j is a first gyroscope 240; the x-ray generator comprises a second gyroscope 240; and the control system is further configured to provide the information related to an orientation of the x-ray generator based on the second gyroscope 240 of the x-ray generator.

In some embodiments, the gyroscope 240 of the x-ray detector 100, 100a-j is a first gyroscope 240; the x-ray generator comprises a second gyroscope 240; and the control system is further configured adjust an orientation of the x-ray detector 100, 100a-j relative to the x-ray generator based on the first gyroscope 240 of the x-ray detector 100, 100a-j and the second gyroscope 240 of the x-ray generator.

In some embodiments, the x-ray system further comprises a camera disposed on the x ray generator; wherein the control system is configured to provide information related to a position of an object to be imaged by the x-ray system based on the camera.

In some embodiments, the control system is configured to provide information based on whether the object has moved.

Although the structures, devices, methods, and systems have been described in accordance with particular embodiments, one of ordinary skill in the art will readily recognize that many variations to the particular embodiments are possible, and any variations should therefore be considered to be within the spirit and scope disclosed herein. Accordingly, many modifications may be made by one of ordinary skill in the art without departing from the spirit and scope of the appended claims.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of the claims beginning with claim [x] and ending with the claim that immediately precedes this one,” where the bracketed term “[x]” is replaced with the number of the most recently recited independent claim. For example, for the first claim set that begins with independent claim 1, claim 4 can depend from either of claims 1 and 3, with these separate dependencies yielding two distinct embodiments; claim 5 can depend from any one of claims 1, 3, or 4, with these separate dependencies yielding three distinct embodiments; claim 6 can depend from any one of claims 1, 3, 4, or 5, with these separate dependencies yielding four distinct embodiments; and so on.

Recitation in the claims of the term “first” with respect to a feature or element does not necessarily imply the existence of a second or additional such feature or element. Elements specifically recited in means-plus-function format, if any, are intended to be construed to cover the corresponding structure, material, or acts described herein and equivalents thereof in accordance with 35 U.S. C. § 112(f). Embodiments of the invention in which an exclusive property or privilege is claimed are defined as follows.

### BACKGROUND

X-ray systems may include automatic exposure control (AEC) components. These AEC components may be mounted in a particular, fixed location. Thus, an operation using the AEC components may be limited to that fixed location.

### BRIEF DESCRIPTION OF THE DRAWINGS

FIG. 1 is a block diagram of a mobile detector including AEC according to some embodiments.

FIG. 2A is a block diagram of a mobile detector including AEC embedded in a front panel according to some embodiments.

FIG. 2B is a block diagram of a mobile detector including AEC behind a front panel according to some embodiments.

FIG. 2C is a block diagram of a mobile detector including AEC behind a front panel according to some embodiments.

FIG. 2D is a block diagram of a mobile detector including AEC behind a front panel according to some embodiments.

FIG. 2E is a block diagram of a mobile detector including AEC behind a front panel with resilient material according to some embodiments.

FIG. 3A is a block diagram of a mobile detector including AEC chamber and an AEC preamplifier according to some embodiments.

FIG. 3B is a block diagram of a mobile detector including AEC chamber coupled to detector circuitry according to some embodiments.

FIG. 4 is a block diagram of a mobile detector including digital AEC according to some embodiments.

FIG. 5 is a block diagram of a mobile detector including AEC and a grid according to some embodiments.

FIG. 6 is a block diagram of an x-ray imaging system according to some embodiments.

FIG. 7 is a block diagram of an x-ray imaging system with multiple detector locations according to some embodiments.

FIG. 8 is a block diagram of an x-ray imaging system with multiple detector locations according to some embodiments.

### DETAILED DESCRIPTION

Conventional fixed installations for radiography applications have a bucky, an automatic exposure control (AEC) chamber and pre-amplifier, and a grid for optimal image quality. When multiple locations are present in the fixed installation, each of those locations will need its own bucky, AEC chamber and pre-amplifier, and grid. For example, a radiology room may include a table and a stand as locations for x-ray imaging. An x-ray generator attached to a crane may be movable to direct x-rays to either location. However, each of the stand and the table will need its own bucky, AEC chamber and pre-amplifier, and grid even if a single detector is moved from location to location.

The fixed installations of AEC chambers prevent the usage of AEC in mobile applications. For example, a patient may be unable to be moved to the radiology room. A mobile detector and x-ray generator may be moved to the patient; however, the system will not have AEC. As a result, AEC and its benefits will not be available when imaging the patient.

As will be described in further detail below, in some embodiments, a mobile detector may eliminate a need for the bucky, AEC chamber, and grid from the system. The AEC chamber and grid functionality may be performed by the mobile detector. In addition to eliminating duplicate parts, embodiments may allow for AEC to be available in mobile applications.

A variety of detectors 100a, 100b, etc. are described below. These detectors may be referred to collectively as detectors 100. A variety of systems 200a, 200b, etc. are described below. These systems may be referred to collectively as systems 200.

FIG. 1 is a block diagram of a mobile detector including AEC according to some embodiments. The detector 100a includes a housing 102, an AEC chamber 104, an imaging array 106, a shield 108, and circuitry 110. The detector 100a and the components are disposed such that imaging array 106 is configured to receive incoming radiation 116 (e.g., incident x-rays) through the AEC chamber.

The housing 102 is a structure enclosing the AEC chamber 104, the imaging array 106, the shield 108, and the circuitry 110. Other components may be enclosed by the housing 102. In some embodiments, the housing 102 fully encloses the various components.

The AEC chamber 104 includes AEC sensors and circuitry configured to transform incoming radiation into an electrical signal or AEC signal. The AEC signals may be used to determine a dose and/or whether to terminate an exposure. The AEC sensors may include solid state sensors, ionization chambers, or the like.

The imaging array 106 includes an array of pixels configured to transform incoming radiation 116 into a two-dimensional image. The imaging array 106 may include direct-conversion sensors, photon counters, indirect-conversion sensors, scintillators, or the like.

The shield 108 is a structure configured to reduce radiation reaching the circuitry 110. For example, the shield 108 may include lead, tin, or other materials that may have a relatively higher absorption of x-rays.

As will be described in further detail below, the AEC chamber 104 including the AEC sensors may be disposed in various locations separate from the imaging array 106, such as in or on a front plate or front panel (not illustrated) of the housing 102, behind the front plate, on the imaging array 106, or the like.

Integration of the AEC sensors with the components of the detector 100a may reduce the costs of the use of an AEC chamber 104. As the AEC chamber 104 is disposed in the detector 100a, when the detector 100a is moved from location to location, such as from a stand in a radiology room to a table, a separate AEC chamber is not needed in those locations. In addition to the reduced number of components, costs and complexity for an original equipment manufacturer (OEM) using the detector 100a may be reduced. An OEM may no longer need to consider buying a separate AEC chamber. An OEM may have a shorter integration time as the detector 100a may have a unified software interface to both the imaging array 106 and the AEC chamber 104. In addition, the detector 100a may be configured to resolve scatter and eliminate a requirement for an external grid.

Moreover, the presence of the AEC chamber 104 in the detector 100a allows AEC to be used in mobile applications outside of the radiology room. AEC in mobile applications may allow for improved workflow, image quality, and lower dose for patients and users, or the like.

In some embodiments, the output of the AEC chamber 104 may be provided to a preamplifier 140. In some embodiments, the preamplifier 140 is disposed outside of the detector 100a. However, in other embodiments, the preamplifier 140 or the function of the preamplifier 140 may be within the detector 100a.

In some embodiments, the housing 102, portions of the housing 102, and other conductive structures, foils, or the like within the housing 102 may form a faraday cage 170 around at least the AEC chamber 104 and up to potentially all internal components of the detector 100a. The AEC chamber 104 may be disposed within the faraday cage 170. The AEC chamber 104 may generate signals that are on the order of millivolts (mV) or microvolts (μV). According, the signals may be particularly susceptible to noise. By placing the AEC chamber 104 within the faraday cage 170, the effect of noise may be reduced. In particular, as will be described in further detail below the preamplifier 140 may be disposed within the faraday cage 170. Thus, the signals from the AEC chamber 104 may be amplified before becoming more susceptible to noise outside of the faraday cage 170.

In some embodiments, the signals from the AEC chamber 104 may be used to begin an exposure. Thus, the AEC chamber 104 may be used to perform an automatic exposure detection (AED) operation. As a result, the detector 100a may not be synchronized with an x-ray generator (not illustrated), but still able to determine the onset of x-rays from the x-ray generator to begin an exposure. This may reduce a cost of a system including the detector 100a, simplify an installation, or the like.

The use of the AEC chamber 104 to begin an exposure may be more robust than using the imaging array 106 to detect x-rays. Sensors within the imaging array 106 may be more sensitive to impacts, mechanical disturbances, or the like. The impacts may result in a false detection of x-rays. An AEC chamber 104 may be less susceptible to such false detections, leading to a more reliable system.

FIG. 2A is a block diagram of a mobile detector including AEC embedded in a front panel according to some embodiments. In some embodiments, the detector 100b may be similar to the detectors 100 described herein. However, the housing 102 includes a front panel 120 (also referred to as a front plate). The front panel 120 may include laminated layers 122 such as carbon fiber layers, fiberglass layers, or the like. AEC sensors 124 of the AEC chamber 104 are disposed within the layers 122 of the front panel 120. In some embodiments, the AEC sensor 124 can be positioned between a top or first layer 122 and a bottom or second layer 122.

In some embodiments, the layers 122 conform to the shape of the AEC sensors 124. However, in other embodiments, a resilient material 126 such as foam may be disposed in substantially the same plane as the AEC sensors 124 and with substantially the same or greater thickness. The resilient material 126 may not be present over the AEC sensors 124. As a result, the AEC sensors 124 may be at least in part isolated from mechanical stresses applied to the front panel 120. For example, a weight of a patient may cause the front panel 120 to flex. The flexing may have a reduced effect on the AEC sensors 124 due to the resilient material 126.

In implementations with a separate AEC chamber, a rigid structure may be present such as a table or a bucky that is between a patient and the AEC chamber. However, with the detector 100b or the like, such rigid structures may not be present. As a result, the detector 100b may include various features as described herein to mitigate flexing that may occur.

FIG. 2B is a block diagram of a mobile detector including AEC behind a front panel according to some embodiments. In some embodiments, the detector 100c may be similar to the detectors 100 described herein. However, the AEC sensors 124 may be disposed behind the front panel 120. In particular, the AEC sensors 124 are disposed on the front panel 120. The AEC sensors 124 may be separated from the imaging array 106 by a gap 128. In some embodiments, the gap 128 may be about 0.2 to about 1 mm or more. In some embodiments, a resilient material 126 may be disposed between the front panel 120 and the imaging array 106.

The resilient material 126 may be disposed around the AEC sensors 124 similar to that described above.

FIG. 2C is a block diagram of a mobile detector including AEC behind a front panel according to some embodiments. In some embodiments, the detector 100d may be similar to the detectors 100 described herein. However, the AEC sensors 124 may be disposed on the imaging array 106. The AEC sensors 124 may be separated from the front panel by a gap 130. In some embodiments, the gap 130 may be about 0.2 to about 1 mm or more. In some embodiments, a resilient material 126 may be disposed between the front panel 120 and the imaging array 106. The resilient material 126 may be disposed around the AEC sensors 124 similar to that described above.

FIG. 2D is a block diagram of a mobile detector including AEC behind a front panel according to some embodiments. In some embodiments, the detector 100e may be similar to the detectors 100 described herein. However, the AEC sensors 124 are disposed within the imaging array 106.

FIG. 2E is a block diagram of a mobile detector including AEC behind a front panel with resilient material according to some embodiments. In some embodiments, the detector 100f may be similar to the detectors 100 described herein. However, the AEC sensors 124 may be disposed between a resilient material 126 and the imaging array 106. The resilient material 126 may be configured to apply an amount of compressive force to the AEC sensors 124 due to compression by the front panel 120. The resilient material 126 may be selected to have composition, thickness, resilience, or the like to apply a predetermined force to the AEC sensors 124. The predetermined force may keep the AEC sensors 124 in place over an expected operation or lifetime of the detector 100f. The predetermined force may be sufficient to keep the AEC sensors 124 in place while being less than an amount that would damage the AEC sensors 124. In addition, the resilient material 126 may be selected to have a lower x-ray attenuation. Examples of the resilient material 126 include foam, rubber, or the like.

In some embodiments, the resilient material 126 may extend across the entire imaging array 106 or beyond the edges of the imaging array 106. In some embodiments, the resilient material 126 may be within one to all of the edges of the imaging array 106 but still overlap with the AEC sensors 124.

While the use of the resilient material 126 is an example of how to maintain the AEC sensors 124 in a particular position, in other embodiments, other techniques may be used. For example, permanent adhesives, releasable adhesives, brackets, clamps, or the like may be used to attach the AEC sensors 124 to the imaging array 106, maintain a relative position between the AEC sensors 124 and the imaging array 106, or the like.

FIG. 3A is a block diagram of a mobile detector including AEC chamber and an AEC preamplifier according to some embodiments. In some embodiments, the detector 100g may be similar to the detectors 100 described herein. The detector 100g includes an AEC preamplifier 140. The preamplifier 140 is disposed within the housing 102. The preamplifier 140 may be disposed within the faraday cage 170. The preamplifier 140 is coupled to the AEC chamber 104 and is configured to receive signals from the AEC chamber 104. In some embodiments, the output of the preamplifier 140 may be a signal 114 that is transmitted to an x-ray generator to control whether the x-ray exposure should stop. Thus, the detector 100g may be a drop-in replacement where a separate AEC chamber may have been used. In some embodiments, the output of the preamplifier 140 may be input to the circuitry 110. AEC signals may be output as signals 112 to a control system, x-ray generator, or the like.

In some embodiments, the preamplifier 140 may be closer to the AEC chamber 104 than would otherwise be achieved with a separate AEC chamber. As a result, the effect of the environment on the signals from the AEC chamber 104 may be reduced, improving signal quality.

FIG. 3B is a block diagram of a mobile detector including AEC chamber coupled to detector circuitry according to some embodiments. In some embodiments, the detector 100h may be similar to the detectors 100 described herein. The AEC chamber 104 is electrically coupled to the circuitry 110. In some embodiments, the AEC chamber 104 may be electrically coupled to the circuitry 110 through pogo pins, cables, wires, transmission lines, or the like.

In some embodiments, the preamplifier 140 is part of the circuitry 110. The circuitry 110 may be configured to perform all of the signal conditioning associated with signals from the AEC chamber 104. The signal 114 or portion of the signals 112 may be in a format suitable for use by an x-ray generator such that the x-ray generator may disable the production of x-rays. In other embodiments, less than all of such signal processing may be performed by the circuitry 110. In some embodiments, the circuitry 110 is configured to generate a digital pulse, a ramp signal, or the like to start and/or stop the exposure using the x-ray generator. In some embodiments, the circuitry 110 may be configured to provide power to the AEC chamber 104, debounce signals from the AEC chamber 104, or the like.

In some embodiments, the circuitry 110 may include comparators, amplifiers, or the like to perform particular operations based on the signals from the AEC chamber 104. For example, one or more signals from the AEC chamber 104 may be compared with a threshold to determine whether to start an exposure. An image may be generated from the imaging array 106 based on the comparison.

In some embodiments, the communication of the AEC signals from the detector 100h may be through a variety of communication techniques. For example, the circuitry 110 may be configured to transmit AEC signals through a low latency digital wireless link, a wired tether with digital and/or analog signals, or the like. The signals 112 are representative of signals transmitted through such media.

In some embodiments, the circuitry 110 is configured to receive configuration inputs to define the AEC configuration. For example, the circuitry 110 may be configured to perform the calibration of the AEC chamber 104.

As described above, the preamplifier 140 may be disposed within the detector 100 and disposed within a faraday cage 170 of the detector. Thus, the signals input to the preamplifier 140 from the AEC chamber 104 may be less susceptible to noise.

FIG. 4 is a block diagram of a mobile detector including a digital AEC 150 according to some embodiments. In some embodiments, the detector 100i may not include a separate physical AEC chamber. The circuitry 110 may be configured to use the imaging array 106 to perform a digital AEC function.

FIG. 5 is a block diagram of a mobile detector including AEC and a grid according to some embodiments. The detector 100j may be similar to the various detectors 100a-100i with AEC as described above, or the like. A grid 160 is disposed on the detector 100j. In some embodiments, the grid 160 includes a series of lead lines extending from one side of the detector 100j to the other. The lead lines may help with backscatter from the detector 100j.

FIG. 6 is a block diagram of an x-ray imaging system according to some embodiments. The system 200a includes a detector 202, a control system 204, an x-ray generator 206, and a computer 208. The detector 202 may include any of the detectors 100 described above or the like. However, in other embodiments, the detector 202 may include different types of detectors.

In some embodiments, the x-ray generator 206 is configurable to project x-rays 212 towards the detector 202. An object 210 such as a patient may be disposed in the path of the x-rays 212 to generate an image. The x-ray generator 206 may include an x-ray tube, power supplies, high voltage generators, or the like.

In some embodiments, the control system 204 may present a unified interface to the computer 208. The system 200a may be installed with a variety of different types of x-ray generators 206, detectors 202, or the like. In some embodiments, if the detector 202 is a detector 100 as described above, the AEC chamber is integrated with the detector 202. The control system 204 may be configured to communicate with and control the detector 202 and the x-ray generator 206. An installer installing the system 200a may only need to configure the computer 208 to communicate with the control system 204. The control system 204 handles the communication and control between the other components of the system 200a. Thus, the installer no longer needs to be concerned with the configuration and interaction between different types of x-ray generators 206 and detectors 202. The control system 204 may present a single interface regardless of the particular type or manufacturer of the x-ray generator 206.

In some embodiments, the AEC signals that would otherwise have been connected to the x-ray generator 206 from a separate AEC chamber (not illustrated) may be instead provided by the control system 204. The control system 204 may interact with the detector 202 to receive the AEC signals in whatever form the detector 202 may provide as described above. The control system 204 may then pass those AEC signals or transform the signals into an appropriate format for the x-ray generator 206 such that the operation of the detector 202 and the x-ray generator 206 may control the dose as desired. For example, the system 200a may have a response time that is faster than about 10 milliseconds (ms) from the x-ray generator of AEC signals to a resulting change in state of the x-ray generator 206. In some embodiments, the control system 204 may be configured to control other aspects of an AEC chamber of the detector 202. For example, the control system 204 may be configured to control calibration of the AEC chamber.

In some embodiments the control system 204 may be configured to synchronize the detector 202 and x-ray generator 206 operation. For example, the control system 204 may be configured to synchronize the generation of x-rays from the x-ray generator 206 and the capture of an image or video by the detector 202 in response to the x-rays. The control system 204 may be configured to synchronize the AEC signals with the x-ray generator 206.

In some embodiments, the detector 202 may be configured to perform grid suppression if a physical grid is present on the detector 202, such as in detector 100j, or in the system 200a. In some embodiments, the detector 202 may be configured to perform lag correction.

In some embodiments, the control system 204 may include a preamplifier 140 configured to amplify AEC signals from an AEC chamber 104 of the detector 202. The preamplifier 140 may operate similarly as described above when integrated with or separate from the detector 202. In some embodiments, a preamplifier 140 may be part of the detector 202 in addition to the preamplifier 140 of the control system 204 and may be the same or different from the preamplifier 140 of the control system 204.

FIG. 7 is a block diagram of an x-ray imaging system with multiple detector locations according to some embodiments. The system 200b may be similar to other systems 200 described herein. A portion of the x-ray system 200b is illustrated and the x-ray system 200b may include other components similar to other x-ray systems 200. In some embodiments, the system 200b may include multiple locations 230. Here two locations 230-1 and 230-2 are illustrated as examples.

In each location 230, a grid 214, an AEC chamber 216, and a bucky 218, illustrated with dashed lines, are not present. The grid 214 and AEC chamber 216, and bucky 218 are illustrated to show the components that are no longer needed for operation. The detector 202 need only be mounted on the corresponding structure, such as a tray, of the location 230. As described above, the detector 202 may include the functionality provided by the grid 214 and the AEC chamber 216 that are not present. For example, location 230-1 may include a stand 220. The detector 202 may be mounted on the stand 220 without a bucky 218. Similarly, the location 230-2 may include a table 222. The detector 202 may be mounted on the table 222 without a bucky 218. Each location 230 may include a simple tray or other structure to hold the detector 202 in place, prevent damage such as scratches on the detector 202 surface, or the like. Moving parts of the bucky 218, shielding for the electronics of the bucky 218, UL certification of the components, or the like may be eliminated. Alignment to the grid 214 may be eliminated. These eliminations of components or processes may reduce costs associated with the system 200b.

The x-ray generator 206 may be mounted on a crane 232 that is configured to move the x-ray generator 206 to be in the orientation appropriate for the particular location 230 where the detector 202 is mounted. The x-ray generator 206 is illustrated in one position with solid lines and an alternate position with dashed lines. Depending on the particular desired application, the detector 202 may be moved between the locations 230 and moved to other locations.

In some embodiments, the detector 202 may be configured to perform scatter correction. As a grid 214 may not be present, including not being present on the detector 202, the detector 202 may be configured to perform scatter correction on the resulting image to reduce or eliminate an effect of the absence of the grid 214. In other embodiments, the control system 204 may be configured to perform the scatter correction on an image or images from the detector 202.

When using a system 200b, the cost and complexity of integrating an AEC chamber, preamplifier, bucky, grid, or the like may be reduced or eliminated.

FIG. 8 is a block diagram of an x-ray imaging system with multiple detector locations according to some embodiments. The system 200c may be similar to other systems 200 described herein. A portion of the x-ray system 200c is illustrated and the x-ray system 200b may include other components similar to other x-ray systems 200. In some embodiments, the detector 202 and the x-ray generator 206 may each include a gyroscope 240. The gyroscope 240 may be configured to determine a relative rotation in one or more axes of the respective device. The gyroscope 240 associated with the x-ray generator 206 may be mounted on x-ray generator 206, on the collimator 252, or the like. In some embodiments, the crane 232 may include sensors 244 configured to sense the relative motion of components of the crane 232. The sensors 244 may be transformed into an orientation of the x-ray generator 206 in place of or in addition to the gyroscope 240.

Using the orientation information of the detector 202 and the x-ray generator 206, the detector 202 and the x-ray generator 206 may be oriented so that an angle 250 between a major axis 246 of the x-rays generated by the x-ray generator 206 and the axis 248 of the detector 202 that is perpendicular to the imaging array 106 may be set as desired or minimized. In some embodiments, the control system 204 may be configured to automatically activate actuators of the crane 232 to orient the x-ray generator 206 so that the axes 246 and 248 are substantially parallel. In other embodiments, the angle 250 or other alignment information may be presented by the control system 204 to an operator of the computer 208. As a result, an image quality may be improved. As the angle 250 between the major axis 246 and the axis 248 increases, an image quality of an image generated by the imaging array 106 may be reduced. Minimizing the angle 250 may increase the image quality. In some embodiments, the angle 250 or information based on the angle may be reported to an operator.

In some embodiments, a camera 242 may be coupled to the x-ray generator 206. The control system 204 may be configured to use an image or video from the camera 242 to determine if a patient moves and inform an operator of the computer 208. In other embodiments, the control system 204 may be configured to use an image or video from the camera 242 to select a portion of the object 210, such as a particular portion of the anatomy of a patient. In other embodiments, the control system 204 may be configured to use an image or video from the camera 242 to determine the source-to-image distance (SID) of the x-ray generator 206 and detector 202. The control system 204 may be configured to use actuators of the crane 232 to achieve a desired SID and/or present such information to an operator of the computer 208 and/or allow the operator to set a desired SID. In some embodiments, the control system 204 may be configured to use adjust a collimator 252 based on an image or video from the camera 242. For example, the control system 204 may be configured to adjust the collimator 252 so that the x-rays emitted from the x-ray generator 206 correspond to the imaging area of the imaging array 106 of the detector 202.

Although various detectors 100, systems 200, or the like have been described above with respect to radiography, other embodiments may be used in fluoroscopic applications.

Some embodiments include an x-ray detector 100, 100a-j, comprising: a housing 102; an imaging array 106 disposed within the housing 102; circuitry 110 configured to generate an image in response to the imaging array 106; and an automatic exposure control (AEC) chamber 104 disposed within the housing 102, separate from the imaging array 106, and on a side of the imaging array 106 opposite to the circuitry 110.

In some embodiments, the housing 102 comprises a front panel 120.

In some embodiments, the AEC chamber 104 is disposed within the front panel 120.

In some embodiments, the x-ray detector further comprises a layer of resilient material 126 disposed within the front panel 120; wherein sensors 124 of the AEC chamber 104 are disposed within a plane of the layer of the resilient material 126 within the front panel 120.

In some embodiments, the AEC chamber 104 is separated from the imaging array 106 by a gap.

In some embodiments, the AEC chamber 104 is disposed on the imaging array 106.

In some embodiments, the x-ray detector further comprises a layer of resilient material 126 disposed between the AEC chamber 104 and the front panel 120.

In some embodiments, the x-ray detector further comprises a shield disposed between the imaging array 106 and the circuitry 110.

In some embodiments, the x-ray detector further comprises an AEC preamplifier 140 coupled to the AEC chamber 104.

In some embodiments, the AEC preamplifier 140 is separate from the circuitry 110.

In some embodiments, the AEC preamplifier 140 is integrated with the circuitry 110.

In some embodiments, the x-ray detector further comprises a faraday cage surrounding at least the AEC chamber 104 and the AEC preamplifier 140.

In some embodiments, the x-ray detector further comprises a grid disposed on an exterior surface of the housing 102.

In some embodiments, the circuitry 110 is configured to perform scatter correction on the image.

In some embodiments, the circuitry 110 is further configured to operate in response to an AEC signal from the AEC chamber 104.

In some embodiments, the circuitry 110 is further configured begin an exposure to generate the image in response to the AEC signal from the AEC chamber 104.

Some embodiments include a method comprising: generating an automatic exposure control (AEC) signal from an AEC chamber 104 within a housing 102 and separate from an imaging array 106 within the housing 102 and generating an image using the imaging array 106 within the housing 102 in response to the AEC signal.

In some embodiments, generating the image comprises beginning an exposure associated with the image in response to the AEC signal.

In some embodiments, generating the image comprises ending an exposure associated with the image in response to the AEC signal.

Some embodiments include an x-ray system, comprising: an x-ray detector 100, 100a-j including an automatic exposure control (AEC) chamber disposed within the x-ray detector 100, 100a-j and separate from an imaging array 106 of the x-ray detector 100, 100a-j; an x-ray generator; and a control system; wherein the control system is configured to: receive AEC signals from the x-ray detector 100, 100a-j; and change an operation of the x-ray generator in response to the AEC signals.

In some embodiments, the control system comprises an AEC preamplifier configured to receive AEC signals from the x-ray detector 100, 100a-j; and the control system is configured to change the operation of the x-ray generator in response to the AEC signals received by the AEC preamplifier.

In some embodiments, the x-ray detector 100, 100a-j comprises any of the x-ray detectors 100, 100a-j of claims 1-16.

In some embodiments, the x-ray generator is movable to project x-rays towards either of a first location and a second location; the x-ray detector 100, 100a-j is movable between the first location and the second location; and the x-ray detector 100, 100a-j is configured to provide AEC signals in response to the AEC chamber 104 in both locations.

In some embodiments, the first location does not include a grid; and the second location does not include a grid.

In some embodiments, the x-ray detector 100, 100a-j further comprises a gyroscope 240 disposed within the x-ray detector 100, 100a-j; and the control system is configured to provide information related to an orientation of the x-ray detector 100, 100a-j relative to the x-ray generator based on the gyroscope 240 of the x-ray detector 100, 100a-j.

In some embodiments, the gyroscope 240 of the x-ray detector 100, 100a-j is a first gyroscope 240; the x-ray generator comprises a second gyroscope 240; and the control system is further configured to provide the information related to an orientation of the x-ray generator based on the second gyroscope 240 of the x-ray generator.

In some embodiments, the gyroscope 240 of the x-ray detector 100, 100a-j is a first gyroscope 240; the x-ray generator comprises a second gyroscope 240; and the control system is further configured adjust an orientation of the x-ray detector 100, 100a-j relative to the x-ray generator based on the first gyroscope 240 of the x-ray detector 100, 100a-j and the second gyroscope 240 of the x-ray generator.

In some embodiments, the x-ray system further comprises a camera disposed on the x-ray generator; wherein the control system is configured to provide information related to a position of an object to be imaged by the x-ray system based on the camera.

In some embodiments, the control system is configured to provide information based on whether the object has moved.

Although the structures, devices, methods, and systems have been described in accordance with particular embodiments, one of ordinary skill in the art will readily recognize that many variations to the particular embodiments are possible, and any variations should therefore be considered to be within the spirit and scope disclosed herein. Accordingly, many modifications may be made by one of ordinary skill in the art without departing from the spirit and scope of the appended claims.

The claims following this written disclosure are hereby expressly incorporated into the present written disclosure, with each claim standing on its own as a separate embodiment. This disclosure includes all permutations of the independent claims with their dependent claims.

Moreover, additional embodiments capable of derivation from the independent and dependent claims that follow are also expressly incorporated into the present written description. These additional embodiments are determined by replacing the dependency of a given dependent claim with the phrase “any of the claims beginning with claim [x] and ending with the claim that immediately precedes this one,” where the bracketed term “[x]” is replaced with the number of the most recently recited independent claim. For example, for the first claim set that begins with independent claim 1, claim 4 can depend from either of claims 1 and 3, with these separate dependencies yielding two distinct embodiments; claim 5 can depend from any one of claims 1, 3, or 4, with these separate dependencies yielding three distinct embodiments; claim 6 can depend from any one of claims 1, 3, 4, or 5, with these separate dependencies yielding four distinct embodiments; and so on.

Recitation in the claims of the term “first” with respect to a feature or element does not necessarily imply the existence of a second or additional such feature or element. Elements specifically recited in means-plus-function format, if any, are intended to be construed to cover the corresponding structure, material, or acts described herein and equivalents thereof in accordance with 35 U.S. C. § 112(f). Embodiments of the invention in which an exclusive property or privilege is claimed are defined as follows.

1. An x-ray detector comprising:
a housing;
an imaging array disposed within the housing;
circuitry configured to generate an image in response to the imaging array; and
an automatic exposure control (AEC) chamber disposed within the housing, separate from the imaging array, and on a side of the imaging array opposite to the circuitry.

2. The x-ray detector of claim 1, wherein:
the housing comprises a front panel; and
the AEC chamber is disposed within the front panel.

3. (canceled)

4. The x-ray detector of claim 2, further comprising:
a layer of resilient material disposed within the front panel;
wherein sensors of the AEC chamber are disposed within a plane of the layer of the resilient material within the front panel.

5. The x-ray detector of claim 1, wherein:
the AEC chamber is separated from the imaging array by a gap.

6. (canceled)

7. The x-ray detector of claim 1, wherein:
the housing comprises a front panel; and
the x-ray detector further comprises a layer of resilient material disposed between the AEC chamber and the front panel.

8. The x-ray detector of claim 1, further comprising:
a shield disposed between the imaging array and the circuitry.

9. (canceled)

10. The x-ray detector of claim 1, further comprising:
an AEC preamplifier coupled to the AEC chamber, wherein the AEC preamplifier is separate from the circuitry

11. The x-ray detector of claim 1, further comprising:
an AEC preamplifier coupled to the AEC chamber, wherein the AEC preamplifier is integrated with the circuitry.

12. The x-ray detector of claim 1, further comprising:
an AEC preamplifier coupled to the AEC chamber, and
a faraday cage surrounding at least the AEC chamber and the AEC preamplifier.

13. The x-ray detector of claim 1, further comprising:
a grid disposed on an exterior surface of the housing.

14. (canceled)

15. The x-ray detector of claim 1, wherein:
the circuitry is further configured to operate in response to an AEC signal from the AEC chamber.

16. (canceled)

17. A method, comprising:
generating an automatic exposure control (AEC) signal from an AEC chamber within a housing and separate from an imaging array within the housing; and
generating an image using the imaging array within the housing in response to the AEC signal.

18. The method of claim 17, wherein:
generating the image comprises beginning an exposure associated with the image in response to the AEC signal.

19. The method of claim 17, wherein:
generating the image comprises ending an exposure associated with the image in response to the AEC signal.

20. An x-ray system, comprising:
an x-ray detector including an automatic exposure control (AEC) chamber disposed within the x-ray detector and separate from an imaging array of the x-ray detector;
an x-ray generator; and
a control system;
wherein the control system is configured to:
receive AEC signals from the x-ray detector; and
change an operation of the x-ray generator in response to the AEC signals.

21. The x-ray system of claim 20, wherein:
the control system comprises an AEC preamplifier configured to receive AEC signals from the x-ray detector; and
the control system is configured to change the operation of the x-ray generator in response to the AEC signals received by the AEC preamplifier.

22. (canceled)

23. The x-ray system of claim 20, wherein:
the x-ray generator is movable to project x-rays towards either of a first location or a second location;
the x-ray detector is movable between the first location and the second location; and
the x-ray detector is configured to provide AEC signals in response to the AEC chamber in the first location and the second location.

24. (canceled)

25. The x-ray system of claim 20, wherein:
the x-ray detector further comprises a gyroscope disposed within the x-ray detector; and
the control system is configured to provide information related to an orientation of the x-ray detector relative to the x-ray generator based on the gyroscope of the x-ray detector.

26. The x-ray system of claim 25, wherein:
the gyroscope of the x-ray detector is a first gyroscope;
the x-ray generator comprises a second gyroscope; and
the control system is further configured to provide the information related to an orientation of the x-ray generator based on the second gyroscope of the x-ray generator.

27. (canceled)

28. The x-ray system of claim 20, further comprising:
a camera disposed on the x-ray generator;
wherein the control system is configured to provide information related to a position of an object to be imaged by the x-ray system based on the camera.

29. (canceled)
